(19) United States
(12) Patent Application Publication
(10) Pub. No.: US 2020/0226483 A1
(43) Pub. Date: Jul. 16, 2020

(54) PROMOTING REFLECTIVE ENGAGEMENT WITH RANKED ALTERNATIVES PRODUCED BY AN ARTIFICIAL INTELLIGENCE SYSTEM

(71) Applicant: International Business Machines Corporation, Armonk, NY (US)

(72) Inventors: Rachel Katherine Emma Bellamy, Bedford, NY (US); Thomas D. Erickson, Minneapollis, MN (US)

(73) Assignee: International Business Machines Corporation, Armonk, NY (US)

(21) Appl. No.: 16/248,927

(22) Filed: Jan. 16, 2019

(51) Int. Cl. G06N 5/04; G06N 3/00; G16H 50/20

(52) U.S. Cl. CPC G06N 5/043; G16H 50/20; G06N 3/006

(57) ABSTRACT

One or more alternatives are received. Each of the one or more alternatives has an associated weight. An advocate-agent corresponding to each of the one or more alternatives is generated in which each advocate-agent is configured to advocate for the corresponding alternative. An interaction characteristic is determined for each advocate-agent based on the associated weight and an engagement model. An engagement metric associated with each advocate-agent is received in which the engagement metric is indicative of an attention level of a user towards the particular alternative associated with the particular advocate-agent. The interaction characteristic a particular advocate-agent is adjusted based upon the engagement metric associated with the particular advocate-agent according to the engagement model.

### TECHNICAL FIELD

The present invention relates generally to a method, system, and computer program product for engagement with an artificial intelligence system. More particularly, the present invention relates to a method, system, and computer program product for promoting reflective engagement with ranked alternatives produced by an artificial intelligence system.

### BACKGROUND

Artificial intelligence (AI) systems are systems that use machine learning and other techniques from artificial intelligence to give the appearance of possessing cognitive properties such as language understanding, memory, learning, attention, and/or reasoning. Often AI systems are designed to work in partnership with human users, enabling the human users to engage in fine-grained, mixed initiative interactions. Examples of AI systems include cognitive learning, recommender, and search engines that return multiple results.

### SUMMARY

The illustrative embodiments provide a method, system, and computer program product. An embodiment of a computer-implemented method includes receiving one or more alternatives. In the embodiment each of the one or more alternative has an associated weight. The embodiment further includes generating an advocate-agent corresponding to each of the one or more alternatives in which each advocate-agent is configured to advocate for the corresponding alternative. The embodiment further includes determining an interaction characteristic for each advocate-agent based on the associated weight and an engagement model. The embodiment further includes receiving an engagement metric associated with each advocate-agent in which the engagement metric is indicative of an attention level of a user towards the particular alternative associated with the particular advocate-agent. The embodiment further includes adjusting the interaction characteristic of a particular advocate-agent based upon the engagement metric associated with the particular advocate-agent according to the engagement model.

Another embodiment further includes determining a proportion of attention from the user for each advocate-agent from a total attention of the user among all advocate-agents based upon the engagement metric for each advocate-agent, and determining that the proportion for the particular advocate-agent does not match an amount specified by the engagement model within a predetermined tolerance value.

In another embodiment, the adjusting of the interaction characteristic of the particular advocate-agent is responsive to the determining that the proportion of attention for the particular advocate-agent does not match within the predetermined tolerance value.

In another embodiment, the interaction characteristic is adjusted corresponding to engagement modulation functions specified by the engagement model.

In another embodiment, adjusting the interaction characteristic the particular advocate-agent includes adjusting one or more of an appearance of the particular advocate-agent or a behavior of the particular advocate-agent.

In another embodiment, the appearance of the particular advocate-agent includes one or more of a size, a render quality, or a style of dress of the particular advocate-agent. In another embodiment, the behavior of the particular advocate-agent includes one or more of a gesture of the particular advocate-agent or a speaking style of the particular advocate-agent.

Another embodiment further includes generating a moderator agent based upon a specification by the engagement model. Another embodiment further includes directing, by the moderator agent, attention of the user to the particular advocate-agent based upon the engagement metric associated with the particular advocate-agent.

In another embodiment, the weight associated with an alternative is based upon a confidence score for the alternative.

An embodiment includes a computer usable program product. The computer usable program product includes one or more computer-readable storage devices, and program instructions stored on at least one of the one or more storage devices.

An embodiment includes a computer system. The computer system includes one or more processors, one or more computer-readable memories, and one or more computer-readable storage devices, and program instructions stored on at least one of the one or more storage devices for execution by at least one of the one or more processors via at least one of the one or more memories.

### BRIEF DESCRIPTION OF THE DRAWINGS

Certain novel features believed characteristic of the invention are set forth in the appended claims. The invention itself, however, as well as a preferred mode of use, further objectives and advantages thereof, will best be understood by reference to the following detailed description of the illustrative embodiments when read in conjunction with the accompanying drawings, wherein:

FIG. 1 depicts a block diagram of a network of data processing systems in which illustrative embodiments may be implemented;

FIG. 2 depicts a block diagram of a data processing system in which illustrative embodiments may be implemented;

FIG. 3 depicts a block diagram of an example configuration for promoting reflective engagement with ranked alternatives produced by an artificial intelligence system in accordance with an illustrative embodiment;

FIG. 4 depicts an example for modulating appearance and behavior of an advocate-agent in accordance with an illustrative embodiment;

FIG. 5 depicts an example user interface with agents represented as graphical avatars in accordance with an illustrative embodiment; and

FIG. 6 depicts a flowchart of an example process for promoting reflective engagement with ranked alternatives produced by an artificial intelligence system in accordance with an illustrative embodiment.

### DETAILED DESCRIPTION

The illustrative embodiments described herein are directed to promoting reflective engagement with ranked alternatives produced by an artificial intelligence (AI) system. One or more embodiments are directed to encouraging users to reflect on and carefully evaluate multiple alternatives provided by an AI system, such as suggested diagnoses, possible courses of treatments, and predictions about future states, in which each alternative has a likelihood, probability, or other ranking associated therewith. Embodiments recognize that one concern regarding interaction of users with existing AI systems is that, especially as AI systems are increasingly used by less expert users, users will tend to treat an ordered list of outcomes as a vote in which the top recipient of votes is the “winner” and the correct outcome. Embodiments recognize that users just picking the top result often results in users missing out on much of the power of AI systems.

An example AI system may draw on analyses of large amounts of data to offer hypotheses about possible diagnoses or courses of treatments for a patient. The example AI system may display results to a user as alternatives in which each alternative is labeled in terms of a confidence rating of the result. Embodiments recognize that a likely failure mode is for the user of such an AI system, especially one who has no background in computer science, to simply choose the alternative with the highest level of confidence (or probability, or other numeric score). This is likely because many non-technical people may be accustomed to elections in which the alternative with the most votes “wins”. Such scenarios may result in a mistake because the set of hypotheses, and their associated levels of confidence, may be the result of a vast amount of knowledge and computation. A user selecting one alternative, and ignoring the rest of the alternatives, may result in the user missing much of the value that such AI systems are capable of providing.

Embodiments recognize that an aim is for users to consider all of the alternatives, the associated confidence levels, and other information, to support reflection by the users, who can bring their own experiences, values and personal knowledge to bear on a problem (e.g., information that the cognitive system does not have access to). Certain embodiments recognize that AI systems should be beginning conversations and decision processes, not necessarily concluding them, and that representing the alternatives as digital entities that can be participants in an interaction provides a means to this end.

Embodiments further recognize that an increasing numbers of AI systems use agents, from chatbots to robots, use language including speech, textual language, signs, or symbols as a means to interact with users. While most current AI systems currently manage only simple responses to questions, or deliver highly structured responses (e.g., wayfinding systems that provide instructions for following a route), considerable research is being done in an effort to produce more conversational systems. Such technologies enable more content-oriented conversations thus may be improved by various embodiments described herein.

Embodiments further recognize that many people may not be adept at reasoning using probabilities or other numerical scores. For example, may people will greatly favor a course of treatment that is said to have a 90% chance of success over one that has a 10% chance of failure, even though the overall probabilities are the same as a form of risk aversion. More generally, it is well documented that humans are subject to a variety of cognitive biases that distort their reasoning processes. An example is the case of risk aversion as just described. Another example is confirmation bias, which is the tendency for people to look only for evidence which confirms a belief or preferred plan, rather than looking for evidence that contradicts it. Therefore, embodiments recognize that it would be valuable for an AI system that presents users with alternatives to provide a mechanism for assisting users in reflectively engaging with the alternatives, rather than simply selecting the “top” alternative.

In an embodiment, a system referred to herein as a reflective engagement system (RES) with respect to various embodiments, receives N alternatives from an AI system as a result of a user interaction with or query of the AI system in which the N alternatives are rank ordered by confidence, likelihood, probability, or some other ranking method. In the embodiment, the RES generates N digital agents in which each digital agent uniquely represents one of the N alternatives. In the embodiment, the RES tailors each agent's appearance and/or behavior to reflect its confidence in the alternative as suggested by the ranking. In the embodiment, as users interact with each agent to better understand its alternative, agents that receive less attention than merited by their associated confidence in their alternatives will engage in various attention-attracting behaviors to increase user engagement with their alternatives. In one or more embodiments, representing alternatives as digital agents, rather than a rank-ordered list of textual descriptors, makes use of psychological and social factors to encourage users to consider multiple alternatives.

In various embodiments, the RES helps communicate that the cognitive system has not arrived at a single solution because there are multiple agents advocating multiple alternatives, and that more than the top alternative is worthy of consideration due to agents representing unconsidered alternatives behaving or appearing in ways that attract more attention to their alternatives. This representation also provides a natural way for users to interact with and learn more about the advantages and disadvantages of the various alternatives, and those interactions are also where agents representing unconsidered alternatives can use their behavior to request attention.

Various embodiments provide for a RES to assist users in critically reflecting on alternatives produced by an AI System, and to encourage the users to consider all alternatives. In an embodiment, the system receives input from an AI system in the form of alternatives with associated rankings such as likelihoods, confidence levels, or probabilities. In the embodiment, the system creates an advocate-agent to represent each alternative. In particular embodiments, agents may be embodied via text (e.g., chatbots), graphics (e.g., visual avatars), sound (e.g., voice-only), and/or tangible objects or media (e.g., robots or other physical devices). Having advocate-agents advocate the alternatives helps underscore the concept that the AI system has not come to a conclusion, contrasting with for instance, an election which would result in agreement on the top vote-receiver.

In the embodiment, an initial appearance and behavior of each advocate-agent reflects the level of confidence or other numerical score associated with the alternative represented by the advocate agent. In particular embodiments, advocate-agents representing higher confidence alternatives are larger, more visible, and/or more behaviorally dominant. As the interaction proceeds, the RES tracks how much attention each advocate-agent is receiving from a user. If some advocate-agents are ignored or receive less attention than is merited by the ranking of their associated alternative, the RES system changes the appearance and behavior of the advocate-agents in ways expected to attract more attention.

In some embodiments, the RES may also create a moderator-agent that manages the interactions among the advocate-agents, and between advocate-agents and users of the RES. In particular embodiments, the moderator-agent acts to direct attention to an advocate-agent that is not receiving enough attention from the user.

In an example scenario according to an embodiment, the RES receives input from an AI system that is configured to present possible courses of treatment for a medical condition with a confidence score associated with each course of treatment. In the example, the AI System offers three courses of treatment, and the RES produces three advocate-agents, portrayed via graphical avatars on a graphical user interface (GUI) of a computer screen, each advocate-agent representing a particular course of treatment.

In the example, the advocate-agent representing the treatment for which the confidence score is highest is displayed to be the largest, speak the loudest, speak the longest, and/or sound the most confident. The advocate-agent may also interrupt the other advocate-agents more than they interrupt it. In the example, the RES correlates the appearances and behaviors with confidence and persuasiveness to attract more attention from users.

In the example, RES includes means of monitoring the amount of attention users direct towards each advocate-agent such as a camera. If the two advocate-agents representing the lower confidence courses of treatment get no attention, the RES may take action to address this situation. For example, the RES may alter the appearance of the two advocate-agents (e.g., making them larger), and/or behavior (e.g. interrupting, speaking more loudly, asking questions), to ensure that two advocate-agents receive more attention from the user and that the user considers the alternatives represented by the advocate-agents. Additionally or alternatively, the RES may create a moderator-agent that acts to direct users' attention to the two under-attended advocate-agents.

In an example interaction for a medical treatment recommendation use case, an AI system includes an on-screen agent with a human-like avatar that interacts with a user via keyboard input and voice input, and lists alternative medical treatments suggest by the AI system. In the example, a RES generates multiple agents, each represented by a human-like avatar that interacts via keyboard and voice to present each alternative to the user.

In the example, a medical professional selects “treatment regimes” in a user interface and a human-like computer agent appears on the screen. The computer agent responds with “There are a large number of treatment regimes, but only 3 of them have success likelihood of greater than 20%. Do you want to focus on those? If not, state how many treatment regimens you would like to hear about, or what the cutoff likelihood should be.” The user responds with “I think three is good”, and the medical professional agrees and instructions the AI system to proceed.

Continuing with the example, the RES causes the original agent to disappear, and in its place, a moderator agent and three advocate-agents appear in which each alternative is represented by a different advocate-agent. The first advocate-agent is small and poorly rendered, and appears more ill at ease than the other agent-advocates. The second agent-advocate is somewhat larger, rendered with a better quality, and dressed in an unassuming beige suit. The third agent-advocate is presented as the largest, is well-rendered, dressed in a nicer suit, and looks confident and decisive. The third agent-advocate may further gesture for attention. Accordingly, an advocate-agent's initial appearance reflects treatment success likelihood, and an advocate-agent's initial behavior, both how it presents itself and how it interacts with the other advocate-agents, reflects treatment success likelihood.

In the example, interaction of the user with the agent-advocates begins. The third, largest agent-advocate having an alternative with an 80% confidence level speaks louder and longer than the other advocate-agents, occasionally interrupts the other advocate-agents, and gestures expansively as it summarizes its recommendation. In a particular example, the first agent-advocate uses eye-tracking to verify that the persons in the room are looking at it as it speaks, and notes when they ask for printouts of some of its information.

Continuing with the example, the second, mid-sized advocate-agent having an alternative with a 50% confidence level speaks in a normal tone of voice, in a plain matter-of-fact manner. Sometimes the second advocate agent may allow the first advocate-agent to interrupt it, other times may not. The second advocate-agent may further make occasional contained gestures. As it speaks, the second advocate-agent uses eye-tracking to verify that those in the room are attentive to it. Although they decline its offer of print-outs, it notes that they pay attention to the offer.

Continuing with the example, the third and smallest advocate-agent speaks softly, sometimes hesitates, may sometimes give way to the other two advocate-agents, and make nervous gestures. In the example, the eye-tracking mechanism indicates that the third advocate-agent is almost entirely ignored while speaking. Accordingly, the RES detects that the people in the room are talking to one another but are not looking at the third advocate-agent, and that its offer of printed materials is ignored.

As the interaction continues, the RES causes the third agent-advocate to grow larger, and/or speak more loudly. The moderator agent requests that the people in the room give third advocate-agent their attention, and they respond. The third advocate-agent offers some critiques of the other alternatives offered, and refuses to be interrupted by the other agents. As t sensing mechanisms detect that the third advocate-agent is receiving more attention, its behavior levels out.

Accordingly, the RES allows an advocate-agent to monitor user responses to its statements and modulates its attempts to get responses. For example, if users have not responded to an advocate-agent, the advocate-agent modulates (e.g., increases) its attempts to get the users to respond to enable the users to consider all alternatives and not to guide them to the most likely solution. In other examples, an advocate-agent not receiving a response may use other procedures to get attention from a user, e.g., an explicit request for consideration. Alternatively, the moderator agent may direct attention of the user to the advocate agent.

In another example, a consumer wishes to obtain a product recommendation from an AI system. In the example, the AI system takes product-preference information from a web-based form for a type of product, analyzes the buying history of others for this type of product, and produces a list of product alternatives that is passed to the RES. In the example, the RES presents an interactive chat window in which for agents are represented via text: a moderator agent and three advocate-agents. In the example, the moderator-agent conducts the interaction, and the advocate-agents each represent one purchase alternative.

Various embodiments described herein describe a reflective engagement system (RES) for promoting reflective engagement with ranked alternatives produced by an artificial intelligence system. In an embodiment, the RES receives input from an AI system that includes weighted alternative to a user query. In the embodiment, the RES uses an optimal engagement model (OEM) to specify a desired engagement model. An engagement model determines how users ought to allocate their attention across the alternatives. An example of an OEM is that the amount of attention directed to an advocate-agent should be proportional to the weight of its alternative divided by the sum of the weights of all alternatives. Although various embodiments are described with respect to receiving ranked alternatives from an AI system, it should be understood that other embodiments may include any system that provides ranked alternatives.

In the embodiment, the RES generates one advocate-agent for each alternative in which the advocate-agent's initial appearance and behavior are adjusted to elicit the amount of attention its alternative ought to receive according to the OEM. In some cases, if specified by the OEM, the RES may also generate a moderator agent to moderate the engagement and interact with the advocate-agents and the users.

In the embodiment, throughout the engagement the RES: (1) maintains and updates engagement metrics as measures of a user's engagement (e.g., responding to a chatbot, eye-tracking to show where the user is looking, sentiment analysis of users responses, clicking, etc.); and (2) uses engagement modulation functions to adjust the appearance and behavior of the advocate-agents so as to optimize the user's engagement with the alternatives.

An embodiment can be implemented as a software application. The application implementing an embodiment can be configured as a modification of an existing application or platform, as a separate application that operates in conjunction with an existing application or platform, a standalone application, or some combination thereof.

The illustrative embodiments are described with respect to certain types of tools and platforms, procedures and algorithms, services, devices, data processing systems, environments, components, AI systems, agents, applications, agent appearances, agent behaviors, interactions, and engagement modulation functions only as examples. Any specific manifestations of these and other similar artifacts are not intended to be limiting to the invention. Any suitable manifestation of these and other similar artifacts can be selected within the scope of the illustrative embodiments.

Furthermore, the illustrative embodiments may be implemented with respect to any type of data, data source, or access to a data source over a data network. Any type of data storage device may provide the data to an embodiment of the invention, either locally at a data processing system or over a data network, within the scope of the invention. Where an embodiment is described using a mobile device, any type of data storage device suitable for use with the mobile device may provide the data to such embodiment, either locally at the mobile device or over a data network, within the scope of the illustrative embodiments.

The illustrative embodiments are described using specific code, designs, architectures, protocols, layouts, schematics, and tools only as examples and are not limiting to the illustrative embodiments. Furthermore, the illustrative embodiments are described in some instances using particular software, tools, and data processing environments only as an example for the clarity of the description. The illustrative embodiments may be used in conjunction with other comparable or similarly purposed structures, systems, applications, or architectures. For example, other comparable mobile devices, structures, systems, applications, or architectures therefor, may be used in conjunction with such embodiment of the invention within the scope of the invention. An illustrative embodiment may be implemented in hardware, software, or a combination thereof.

The examples in this disclosure are used only for the clarity of the description and are not limiting to the illustrative embodiments. Additional data, operations, actions, tasks, activities, and manipulations will be conceivable from this disclosure and the same are contemplated within the scope of the illustrative embodiments.

Any advantages listed herein are only examples and are not intended to be limiting to the illustrative embodiments. Additional or different advantages may be realized by specific illustrative embodiments. Furthermore, a particular illustrative embodiment may have some, all, or none of the advantages listed above.

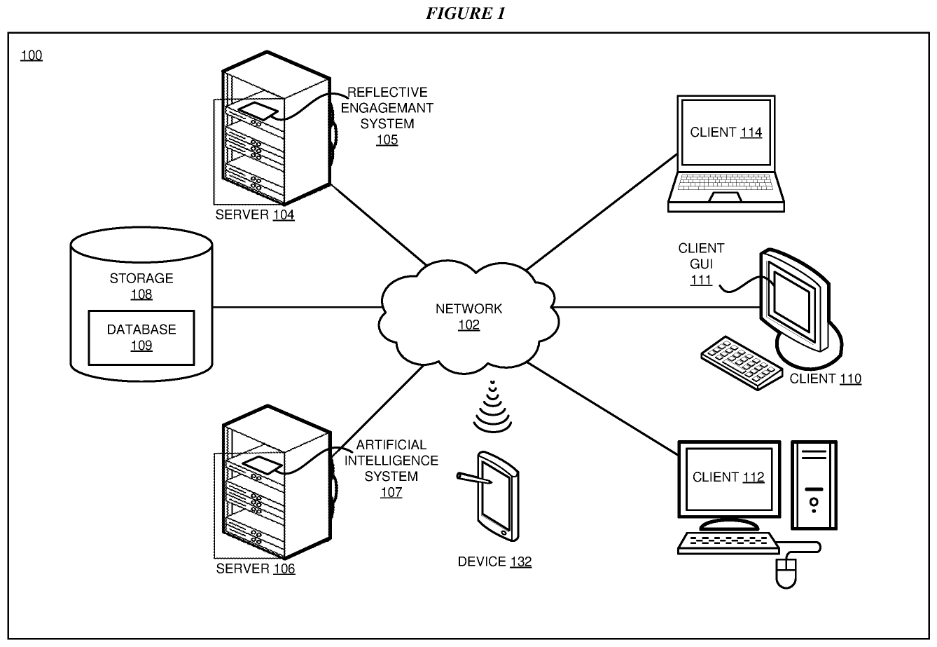

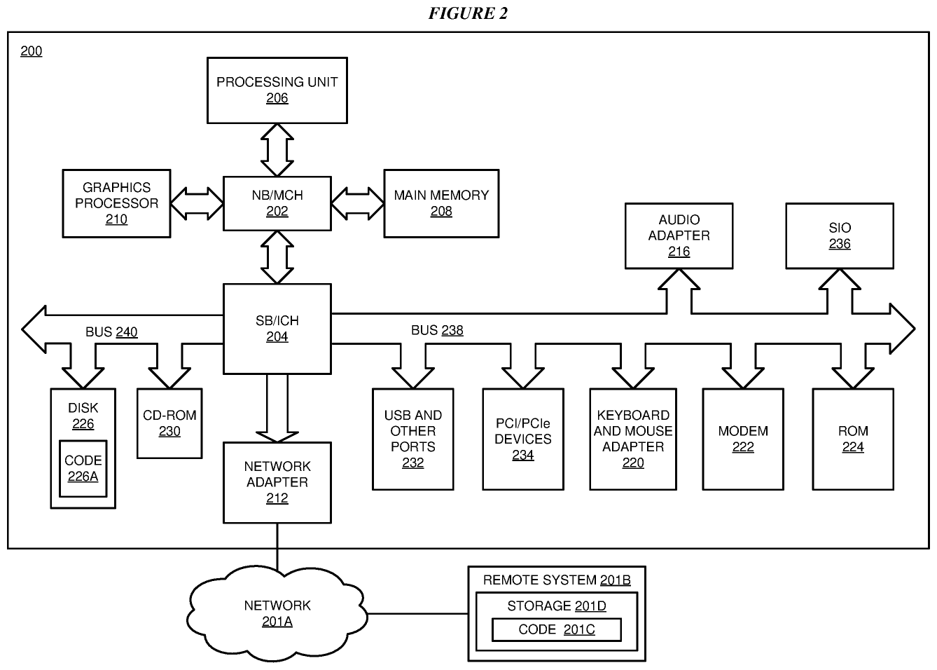

With reference to the figures and in particular with reference to FIGS. 1 and 2, these figures are example diagrams of data processing environments in which illustrative embodiments may be implemented. FIGS. 1 and 2 are only examples and are not intended to assert or imply any limitation with regard to the environments in which different embodiments may be implemented. A particular implementation may make many modifications to the depicted environments based on the following description.

FIG. 1 depicts a block diagram of a network of data processing systems in which illustrative embodiments may be implemented. Data processing environment 100 is a network of computers in which the illustrative embodiments may be implemented. Data processing environment 100 includes network 102. Network 102 is the medium used to provide communications links between various devices and computers connected together within data processing environment 100. Network 102 may include connections, such as wire, wireless communication links, or fiber optic cables.

Clients or servers are only example roles of certain data processing systems connected to network 102 and are not intended to exclude other configurations or roles for these data processing systems. Server 104 and server 106 couple to network 102 along with storage device 108. Software applications may execute on any computer in data processing environment 100. Clients 110, 112, and 114 are also coupled to network 102. A data processing system, such as server 104 or 106, or client 110, 112, or 114 may contain data and may have software applications or software tools executing thereon.

Only as an example, and without implying any limitation to such architecture, FIG. 1 depicts certain components that are usable in an example implementation of an embodiment. For example, servers 104 and 106, and clients 110, 112, 114, are depicted as servers and clients only as example and not to imply a limitation to a client-server architecture. As another example, an embodiment can be distributed across several data processing systems and a data network as shown, whereas another embodiment can be implemented on a single data processing system within the scope of the illustrative embodiments. Data processing systems 104, 106, 110, 112, and 114 also represent example nodes in a cluster, partitions, and other configurations suitable for implementing an embodiment.

Device 132 is an example of a device described herein. For example, device 132 can take the form of a smartphone, a tablet computer, a laptop computer, client 110 in a stationary or a portable form, a wearable computing device, or any other suitable device. Any software application described as executing in another data processing system in FIG. 1 can be configured to execute in device 132 in a similar manner. Any data or information stored or produced in another data processing system in FIG. 1 can be configured to be stored or produced in device 132 in a similar manner.

Servers 104 and 106, storage device 108, and clients 110, 112, and 114, and device 132 may couple to network 102 using wired connections, wireless communication protocols, or other suitable data connectivity. Clients 110, 112, and 114 may be, for example, personal computers or network computers.

In the depicted example, server 104 may provide data, such as boot files, operating system images, and applications to clients 110, 112, and 114. Clients 110, 112, and 114 may be clients to server 104 in this example. Clients 110, 112, 114, or some combination thereof, may include their own data, boot files, operating system images, and applications. Data processing environment 100 may include additional servers, clients, and other devices that are not shown. Server 104 includes a reflective engagement system (RES) 105 that may be configured to implement one or more of the functions described herein for promoting reflective engagement with ranked alternatives produced by an artificial intelligence system in accordance with one or more embodiments. Server 106 includes an artificial intelligence (AI) system 107 for providing ranked alternatives to RES 105 as described herein with respect to various embodiments. In an embodiment, client 110 includes a client graphical user interface (GUI) 111 to enable a user to interact with reflective engagement system 105 and/or AI system 107 as described herein with respect to various embodiments.

Storage device 108 includes a database 109 configured to store one or more of optimal engagement models (OEMs) and weighted appearances/behavioral dimensions as described herein with respect to one or more embodiments.

In the depicted example, data processing environment 100 may be the Internet. Network 102 may represent a collection of networks and gateways that use the Transmission Control Protocol/Internet Protocol (TCP/IP) and other protocols to communicate with one another. At the heart of the Internet is a backbone of data communication links between major nodes or host computers, including thousands of commercial, governmental, educational, and other computer systems that route data and messages. Of course, data processing environment 100 also may be implemented as a number of different types of networks, such as for example, an intranet, a local area network (LAN), or a wide area network (WAN). FIG. 1 is intended as an example, and not as an architectural limitation for the different illustrative embodiments.

Among other uses, data processing environment 100 may be used for implementing a client-server environment in which the illustrative embodiments may be implemented. A client-server environment enables software applications and data to be distributed across a network such that an application functions by using the interactivity between a client data processing system and a server data processing system. Data processing environment 100 may also employ a service oriented architecture where interoperable software components distributed across a network may be packaged together as coherent business applications. Data processing environment 100 may also take the form of a cloud, and employ a cloud computing model of service delivery for enabling convenient, on-demand network access to a shared pool of configurable computing resources (e.g. networks, network bandwidth, servers, processing, memory, storage, applications, virtual machines, and services) that can be rapidly provisioned and released with minimal management effort or interaction with a provider of the service.

With reference to FIG. 2, this figure depicts a block diagram of a data processing system in which illustrative embodiments may be implemented. Data processing system 200 is an example of a computer, such as servers 104 and 106, or clients 110, 112, and 114 in FIG. 1, or another type of device in which computer usable program code or instructions implementing the processes may be located for the illustrative embodiments.

Data processing system 200 is also representative of a data processing system or a configuration therein, such as device 132 or server 104 in FIG. 1 in which computer usable program code or instructions implementing the processes of the illustrative embodiments may be located. Data processing system 200 is described as a computer only as an example, without being limited thereto. Implementations in the form of other devices, such as device 132 in FIG. 1, may modify data processing system 200, such as by adding a touch interface, and even eliminate certain depicted components from data processing system 200 without departing from the general description of the operations and functions of data processing system 200 described herein.

In the depicted example, data processing system 200 employs a hub architecture including North Bridge and memory controller hub (NB/MCH) 202 and South Bridge and input/output (I/O) controller hub (SB/ICH) 204. Processing unit 206, main memory 208, and graphics processor 210 are coupled to North Bridge and memory controller hub (NB/MCH) 202. Processing unit 206 may contain one or more processors and may be implemented using one or more heterogeneous processor systems. Processing unit 206 may be a multi-core processor. Graphics processor 210 may be coupled to NB/MCH 202 through an accelerated graphics port (AGP) in certain implementations.

In the depicted example, local area network (LAN) adapter 212 is coupled to South Bridge and I/O controller hub (SB/ICH) 204. Audio adapter 216, keyboard and mouse adapter 220, modem 222, read only memory (ROM) 224, universal serial bus (USB) and other ports 232, and PCl/PCIe devices 234 are coupled to South Bridge and I/O controller hub 204 through bus 238. Hard disk drive (HDD) or solid-state drive (SSD) 226 and CD-ROM 230 are coupled to South Bridge and I/O controller hub 204 through bus 240. PCl/PCIe devices 234 may include, for example, Ethernet adapters, add-in cards, and PC cards for notebook computers. PCI uses a card bus controller, while PCIe does not. ROM 224 may be, for example, a flash binary input/output system (BIOS). Hard disk drive 226 and CD-ROM 230 may use, for example, an integrated drive electronics (IDE), serial advanced technology attachment (SATA) interface, or variants such as external-SATA (eSATA) and micro-SATA (mSATA). A super I/O (SIO) device 236 may be coupled to South Bridge and I/O controller hub (SB/ICH) 204 through bus 238.

Memories, such as main memory 208, ROM 224, or flash memory (not shown), are some examples of computer usable storage devices. Hard disk drive or solid state drive 226, CD-ROM 230, and other similarly usable devices are some examples of computer usable storage devices including a computer usable storage medium.

An operating system runs on processing unit 206. The operating system coordinates and provides control of various components within data processing system 200 in FIG. 2. The operating system may be a commercially available operating system for any type of computing platform, including but not limited to server systems, personal computers, and mobile devices. An object oriented or other type of programming system may operate in conjunction with the operating system and provide calls to the operating system from programs or applications executing on data processing system 200.

Instructions for the operating system, the object-oriented programming system, and applications or programs, such as application 105 in FIG. 1, are located on storage devices, such as in the form of code 226A on hard disk drive 226, and may be loaded into at least one of one or more memories, such as main memory 208, for execution by processing unit 206. The processes of the illustrative embodiments may be performed by processing unit 206 using computer implemented instructions, which may be located in a memory, such as, for example, main memory 208, read only memory 224, or in one or more peripheral devices.

Furthermore, in one case, code 226A may be downloaded over network 201A from remote system 201B, where similar code 201C is stored on a storage device 201D. In another case, code 226A may be downloaded over network 201A to remote system 201B, where downloaded code 201C is stored on a storage device 201D.

The hardware in FIGS. 1-2 may vary depending on the implementation. Other internal hardware or peripheral devices, such as flash memory, equivalent non-volatile memory, or optical disk drives and the like, may be used in addition to or in place of the hardware depicted in FIGS. 1-2. In addition, the processes of the illustrative embodiments may be applied to a multiprocessor data processing system.

In some illustrative examples, data processing system 200 may be a personal digital assistant (PDA), which is generally configured with flash memory to provide non-volatile memory for storing operating system files and/or user-generated data. A bus system may comprise one or more buses, such as a system bus, an I/O bus, and a PCI bus. Of course, the bus system may be implemented using any type of communications fabric or architecture that provides for a transfer of data between different components or devices attached to the fabric or architecture.

A communications unit may include one or more devices used to transmit and receive data, such as a modem or a network adapter. A memory may be, for example, main memory 208 or a cache, such as the cache found in North Bridge and memory controller hub 202. A processing unit may include one or more processors or CPUs.

The depicted examples in FIGS. 1-2 and above-described examples are not meant to imply architectural limitations. For example, data processing system 200 also may be a tablet computer, laptop computer, or telephone device in addition to taking the form of a mobile or wearable device.

Where a computer or data processing system is described as a virtual machine, a virtual device, or a virtual component, the virtual machine, virtual device, or the virtual component operates in the manner of data processing system 200 using virtualized manifestation of some or all components depicted in data processing system 200. For example, in a virtual machine, virtual device, or virtual component, processing unit 206 is manifested as a virtualized instance of all or some number of hardware processing units 206 available in a host data processing system, main memory 208 is manifested as a virtualized instance of all or some portion of main memory 208 that may be available in the host data processing system, and disk 226 is manifested as a virtualized instance of all or some portion of disk 226 that may be available in the host data processing system. The host data processing system in such cases is represented by data processing system 200.

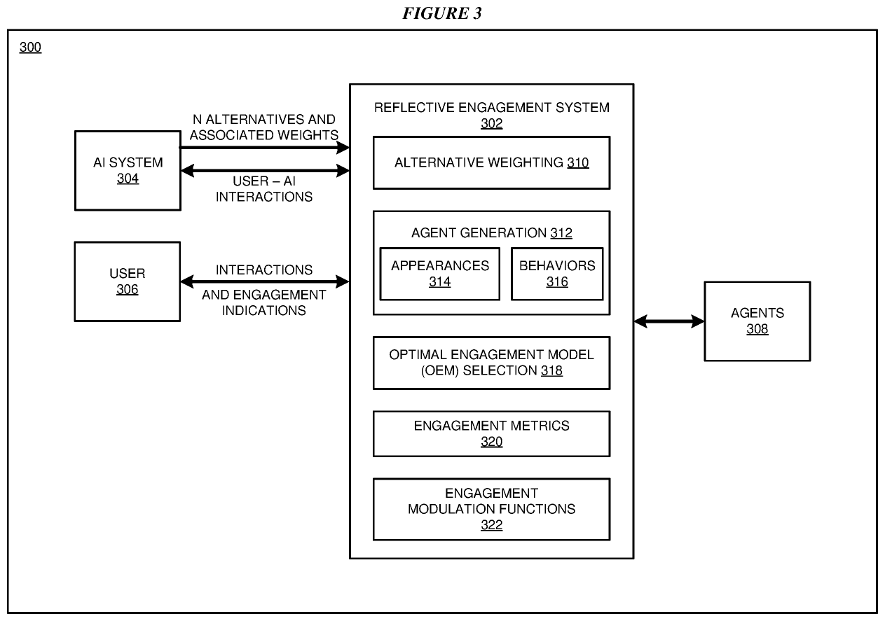

With reference to FIG. 3, this figure depicts a block diagram of an example configuration 300 for promoting reflective engagement with ranked alternatives produced by an artificial intelligence system in accordance with an illustrative embodiment. The example embodiment includes a reflective engagement system (RES) 302. In a particular embodiment, RES 302 is an example of RES 105 of FIG. 1.

RES 302 is configured to receive alternatives with associated weights and user-AI interactions from an AI system 304, and user interactions and engagement indications from a user 306. RES 302 is further configured to generate one or more agents 308 including one or more advocate-agents or moderator agents as described herein. In the embodiment, RES 302 includes an alternative weighting component 310, an agent generation component 312 having an appearances component 314 and a behaviors component 316, an optimal engagement model (OEM) selection component 318, engagement metrics 320, and engagement modulation functions 322.

In the embodiment, alternative weighting component 310 is configured to receive a set of N alternatives A (1. . . N) and associated weights generated by AI system 304 and determine an alternate weight of each alternative such that each alternative, Ai, has a weight, Wi, that is associated with its likelihood, probability, confidence level, or other quantitative metric. In a particular embodiment, a weight is a function of a confidence score. In a particular example of a product recommendation scenario, a weight may be a function of an amount of money that an advertiser pays to have attention director to a product and the confidence score.

Agent generation component 312 is configured to generate advocate-agents for each alternative Ai and a moderator agent if necessary. In one or more embodiments, an advocate-agent is a digital agent that may be represented in one of many forms (e.g., chatbot, graphical avatar, robot, etc.) and capable of interacting with users (e.g. via text, speech, graphics, etc.), and able to detect when the advocate-agent is receiving attention from a human such as user 306 (e.g., via a textual response, button click, vocal response, eye gaze, gesture, etc.). In one or more embodiments, each advocate-agent advocates for a single alternative. A moderator agent is a digital agent, with the same or similar capabilities as an advocate-agent, that is not associated with an alternative, and that serves as a moderator, initiating and ending sessions, and directing the attention of users to particular advocate-agents at particular times.

Appearances component 314 is configured to determine agent appearance characteristics indicating the perceptible representation of the agent. For a graphical screen-based agent, the appearance characteristics may include one or more of size; height; neatness; professional role; professional dress or other style of dress; posture; degree of anthropomorphism; or realism or other render quality of the agent's appearance. For a textual agent (e.g., a chatbot), the appearance characteristics may include one or more of the above appearance characteristics described in text form as well as visual characteristics of the text such as font, font size, etc.

It should be noted that various dimensions of appearance have conventional associations with a degree to which a user will engage (e.g., attention, confidence, trust). For instance, someone who appears to be in a role associated with expertise, and who is neatly dressed in professional attire, will usually be listened to with greater confidence than someone who does not present those attributes. Similarly, people with various physical characteristics may have their opinions taken more seriously and meet with fewer objections.

Behavior component 316 is configured to determine behavioral characteristics of an agent indicating a manner in which the agent behaves during an interaction with user 306. For a graphical screen-based agent, behavioral characteristics may include speech volume; speech smoothness; gaze directness; gesture expansiveness; or tendency to interrupt others; etc. For a textual agent, behavioral characteristics may include the above-described characteristics described in text form (or expressed through dialog), or as expressed through the visual characteristics of the text such as font, font size, etc.

It should be understood that such behavioral characteristics often are understood to reflect confidence, e.g., someone one speaking loudly, and smoothly, and gazing directly at the audience is seen as more confident and knowledgeable than someone speaking softly and failing to make eye contact.

Optimal engagement model (OEM) 318 specifies a desired engagement model, that is, how much attention the user ought to pay to each advocate-agent. An example of OEM 318 is that the relative engagement with an agent ought to be proportional the relative weight of its recommendation, i.e., the advocate-agent with the highest weighted recommendation will receive the most engagement, etc.

Engagement metrics 320 are observable measures of where a user is allocating attention with respect to an advocate-agent. For instance, in the case of a visual, screen-based advocate-agent, if the user looks towards the agent as it is speaking, nods as it is speaking, asks it questions after it is done, or responds to an agent's offer to print out details, these cues may be taken as indications that the user is engaged with the agent (i.e. attending to its alternative). In the case of a textual agent, a user might direct the user's gaze at the text the advocate-agent is generating, respond by typing back, click a button that the agent displays, etc.

Engagement modulation functions 322 are functions that alter an agent's appearance or behavior in ways that that will increase or decrease the amount of attention a user is giving to the agent based upon engagement metrics 320 and the OEM. Examples of appearance dimensions that can be modulated are realism, anthropomorphism, height, manner of dress, or professional role. Examples of behavioral dimensions that can be modulated include volume of speech, amount of gesturing, amount of time speaking, tendency to interrupt others, etc. Another type of behavioral modulation is that the moderator agent might request that users attend to a particular advocate-agent, and may direct that advocate-agent to speak.

Thus, if RES 302 determines in accordance with the OEM to increase the attention an advocate-agent is receiving from user 306, the moderator agent might direct the user's attention to a particular advocate-agent, and RES 302 may use engagement modulation functions 322 to increase the height (appearance) of the advocate-agent, alter a speaking style such as speaking in a louder voice(or use a larger font size or all capital letters for a chatbot), gesture more, interrupt other advocate-agents more, etc. The appearances and behavioral dimensions of other advocate-agents might similarly be adjusted to decrease the amount of attention the advocate-agents receive by shrinking in size, speaking more softly or tentatively, morphing into a less professional appearance, etc.

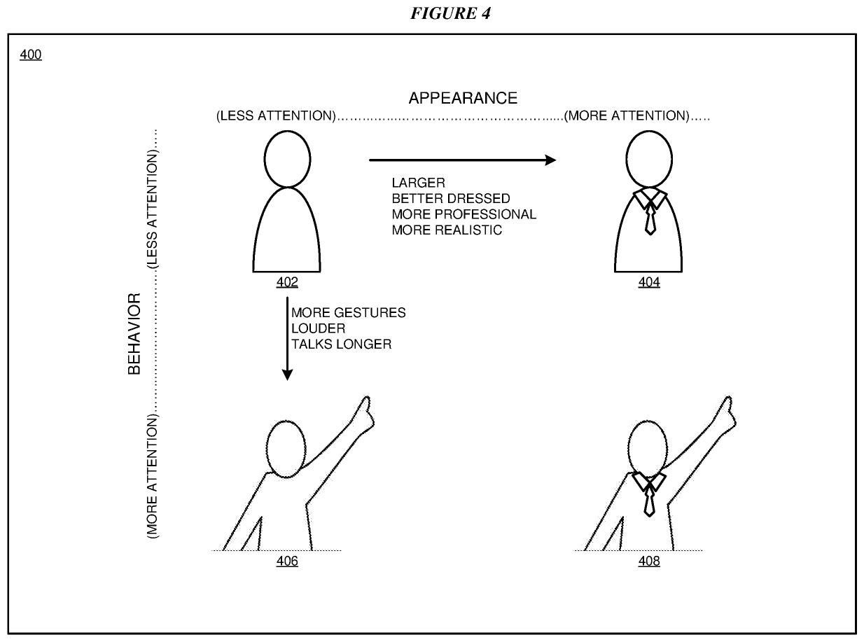

With reference to FIG. 4, this figure depicts an example 400 for modulating appearance and behavior of an advocate-agent in accordance with an illustrative embodiment. FIG. 4 illustrates representations of appearances and behaviors of an advocate-agent based upon an amount of attention that a user is expected to exhibit towards the advocate-agent based upon an OEM. A first advocate-agent representation 402 representing an alternative having an associated relatively lower confidence score for attracting less user attention is shown as simply rendered exhibiting no gestures. A second advocate-agent representation 404 for attracting more user attention is shown as better dressed, more professional, and more realistic but exhibiting no gestures.

A third advocate-agent representation 406 for attracting more user attention is shown as simply rendered but exhibiting a gesture. A fourth advocate-agent representation 408 for attracting still more user attention is shown as better dressed, more professional, and more realistic and exhibiting a gesture. Accordingly, in one or more embodiments, an appearance and/or behavior of an advocate-agent may be modulated to garner more attention from users during an interaction with an AI system.

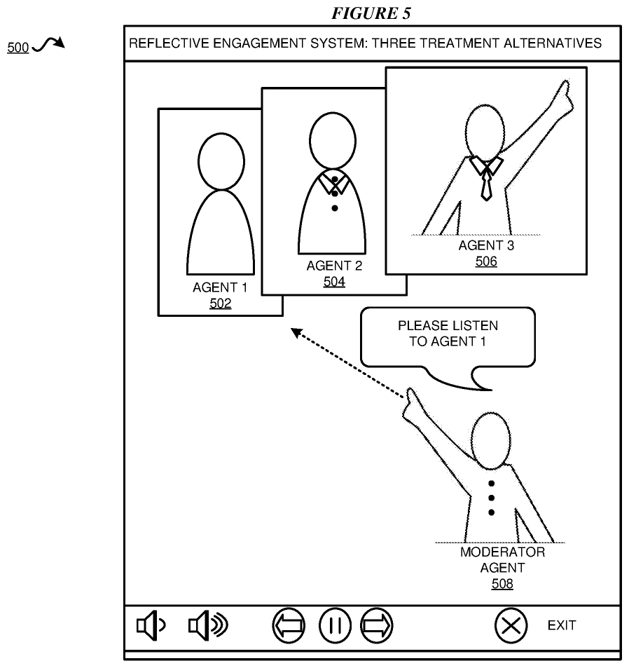

With reference to FIG. 5, this figure depicts an example user interface 500 with agents represented as graphical avatars in accordance with an illustrative embodiment. User interface 500 shows a first advocate-agent 502, a second advocate-agent 504, a third advocate-agent 506, and a moderator agent 508. Each of first advocate-agent 502, second advocate-agent 504, a third advocate-agent 506 each advocate an alternative for a medical treatment.

First advocate-agent 502 advocates for an alternative having an associated confidence score lower than those of second advocate-agent 504 and third advocate-agent 506. First advocate-agent 502 is shown as simply rendered exhibiting no gestures. Second advocate-agent 504 advocates for an alternative having an associated confidence score lower than that of third advocate-agent 506. Second advocate-agent 504 is shown as better dressed, more professional, and more realistic than first advocate-agent 502 but exhibiting no gestures. Third advocate-agent 506 advocates for an alternative having an associated confidence score higher than those of first advocate-agent 504 and second advocate-agent 504. Third advocate-agent 506 is shown as better dressed, more professional, and more realistic than first advocate-agent 502 and exhibiting a gesture.

In the example of FIG. 5, the RES determines that the user is not providing enough attention to the alternative advocated by first advocate-agent 502, and instructs moderator agent 508 to direct the attention of the user towards first advocate-agent 502 by gesturing towards first advocate-agent 502 and saying “Please listen to agent 1”.

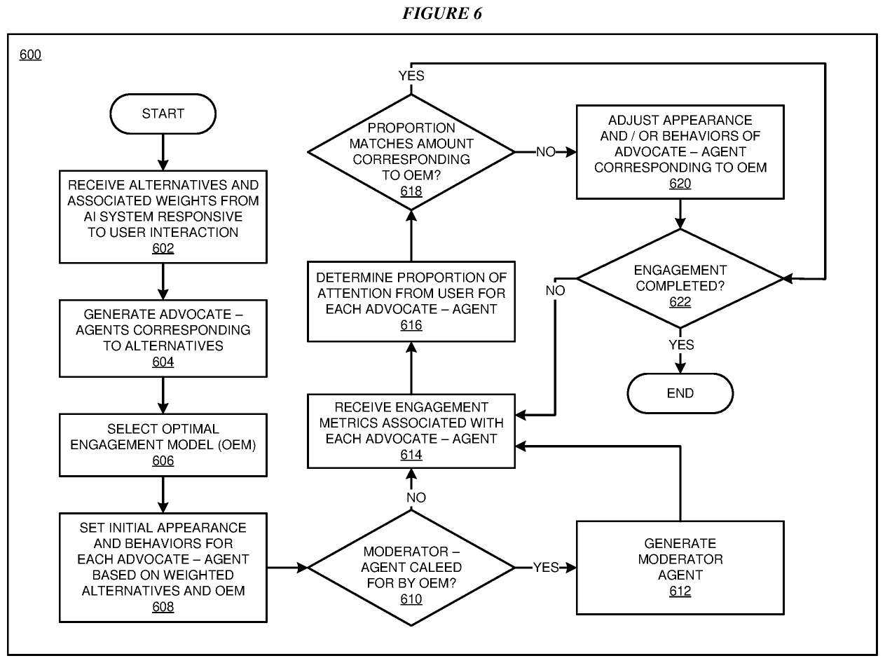

With reference to FIG. 6, this figure depicts a flowchart of an example process 600 for promoting reflective engagement with ranked alternatives produced by an artificial intelligence system in accordance with an illustrative embodiment. In block 602, RES 105 receives one or more alternatives and associated weights from an AI system responsive to a user interaction with the AI system. In block 604, RES 105 generates advocate agents corresponding to each of the one or more alternatives. In block 606, RES 105 selects an optimal engagement model (OEM). In a particular embodiment, the selected OEM adjusts one or more of an appearance and behavior for each advocate-agent based upon the proportion of the weight of the alternative the advocate-agent represents compared to the weights of all advocate-agents.

In block 608, RES 105 sets initial appearance and behaviors (or other interaction characteristic) for each advocate-agent based on the weighted alternative and selected OEM. In particular embodiments, RES 105 selects the weighted appearance and behavior dimensions of an advocate-agent to correspond with the selected OEM. In block 610, RES 105 determines whether a moderator agent is called for by the selected OEM to moderate among the one or more advocate-agents. If RES 105 determines that the selected OEM calls for a moderator agent, in block 612 RES 105 generates a moderator agent and process 600 continues to block 614. If RES 105 determines that the selected OEM does not call for a moderator agent, process 600 continues to block 614.

In block 614, RES 105 receives an engagement metric associated with each advocate-agent in which the engagement metric is indicative of a level of attention of the user towards the particular alternative associated with the particular advocate-agent. In block 616, RES 105 determines a proportion of attention from the user for each advocate-agent from a total attention of the user among all advocate-agents based upon the engagement metric for each advocate-agent.

In block 618, RES 105 determines whether the proportion for an advocate-agent matches an amount corresponding to an amount specified by the OEM. If RES 105 determines that the proportion does not match the amount specified by the OEM within a predetermined tolerance value, in block 620 RES 105 adjusts an appearance and/or behavior dimensions (or other interaction characteristic) of the advocate-agent corresponding to engagement modulation functions specified by the selected OEM and process 600 continues to block 622. In an embodiment, the moderator agent may direct attention of the user to the particular advocate-agent as an alternative to, or in addition to, adjusting the appearance and/or behavior dimensions of the particular advocate-agent based upon the engagement metrics. If RES 105 determines that the proportion does not match the amount specified by the OEM within the predetermined tolerance value, process 600 continues to block 622.

In block 622, RES 105 determines if the engagement between the AI system and the user is completed. If RES 105 determines that the engagement is not completed, process 600 returns to block 614. If RES 105 determines that the engagement is completed, process 600 then ends.

Thus, a computer implemented method, system or apparatus, and computer program product are provided in the illustrative embodiments for promoting reflective engagement with ranked alternatives produced by an artificial intelligence system and other related features, functions, or operations. Where an embodiment or a portion thereof is described with respect to a type of device, the computer implemented method, system or apparatus, the computer program product, or a portion thereof, are adapted or configured for use with a suitable and comparable manifestation of that type of device.

Where an embodiment is described as implemented in an application, the delivery of the application in a Software as a Service (SaaS) model is contemplated within the scope of the illustrative embodiments. In a SaaS model, the capability of the application implementing an embodiment is provided to a user by executing the application in a cloud infrastructure. The user can access the application using a variety of client devices through a thin client interface such as a web browser (e.g., web-based e-mail), or other light-weight client-applications. The user does not manage or control the underlying cloud infrastructure including the network, servers, operating systems, or the storage of the cloud infrastructure. In some cases, the user may not even manage or control the capabilities of the SaaS application. In some other cases, the SaaS implementation of the application may permit a possible exception of limited user-specific application configuration settings.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

What is claimed is:

1. A computer-implemented method comprising:
receiving one or more alternatives, each of the one or more alternative having an associated weight;
generating an advocate-agent corresponding to each of the one or more alternatives, each advocate-agent being configured to advocate for the corresponding alternative;
determining an interaction characteristic for each advocate-agent based on the associated weight and an engagement model;
receiving an engagement metric associated with each advocate-agent, the engagement metric being indicative of an attention level of a user towards the particular alternative associated with the particular advocate-agent; and
adjusting the interaction characteristic of a particular advocate-agent based upon the engagement metric associated with the particular advocate-agent according to the engagement model.

2. The computer-implemented method of claim 1, further comprising:
determining a proportion of attention from the user for each advocate-agent from a total attention of the user among all advocate-agents based upon the engagement metric for each advocate-agent; and
determining that the proportion for the particular advocate-agent does not match an amount specified by the engagement model within a predetermined tolerance value.

3. The computer-implemented method of claim 2, wherein the adjusting of the interaction characteristic of the particular advocate-agent is responsive to the determining that the proportion of attention for the particular advocate-agent does not match within the predetermined tolerance value.

4. The computer-implemented method of claim 2, wherein the interaction characteristic is adjusted corresponding to engagement modulation functions specified by the engagement model.

5. The computer-implemented method of claim 1, wherein adjusting the interaction characteristic the particular advocate-agent includes adjusting one or more of an appearance of the particular advocate-agent or a behavior of the particular advocate-agent.

6. The computer-implemented method of claim 5, wherein the appearance of the particular advocate-agent includes one or more of a size, a render quality, or a style of dress of the particular advocate-agent.

7. The computer-implemented method of claim 5, wherein the behavior of the particular advocate-agent includes one or more of a gesture of the particular advocate-agent or a speaking style of the particular advocate-agent.

8. The computer-implemented method of claim 1, further comprising:
generating a moderator agent based upon a specification by the engagement model.

9. The computer-implemented method of claim 8, further comprising:
directing, by the moderator agent, attention of the user to the particular advocate-agent based upon the engagement metric associated with the particular advocate-agent.

10. The computer-implemented method of claim 1, wherein the weight associated with an alternative is based upon a confidence score for the alternative.

11. A computer usable program product comprising one or more computer-readable storage devices, and program instructions stored on at least one of the one or more storage devices, the stored program instructions comprising:
program instructions to receive one or more alternatives, each of the one or more alternative having an associated weight;
program instructions to generate an advocate-agent corresponding to each of the one or more alternatives, each advocate-agent being configured to advocate for the corresponding alternative;
program instructions to determine an interaction characteristic for each advocate-agent based on the associated weight and an engagement model;
program instructions to receive an engagement metric associated with each advocate-agent, the engagement metric being indicative of an attention level of a user towards the particular alternative associated with the particular advocate-agent; and
program instructions to adjust the interaction characteristic of a particular advocate-agent based upon the engagement metric associated with the particular advocate-agent according to the engagement model.

12. The computer usable program product of claim 11, further comprising:
program instructions to determine a proportion of attention from the user for each advocate-agent from a total attention of the user among all advocate-agents based upon the engagement metric for each advocate-agent; and
program instructions to determine that the proportion for the particular advocate-agent does not match an amount specified by the engagement model within a predetermined tolerance value.

13. The computer usable program product of claim 12, wherein the adjusting of the interaction characteristic of the particular advocate-agent is responsive to the determining that the proportion of attention for the particular advocate-agent does not match within the predetermined tolerance value.

14. The computer usable program product of claim 12, wherein the interaction characteristic is adjusted corresponding to engagement modulation functions specified by the engagement model.

15. The computer usable program product of claim 11, wherein adjusting the interaction characteristic the particular advocate-agent includes adjusting one or more of an appearance of the particular advocate-agent or a behavior of the particular advocate-agent.

16. The computer usable program product of claim 15, wherein the appearance of the particular advocate-agent includes one or more of a size, a render quality, or a style of dress of the particular advocate-agent.

17. The computer usable program product of claim 15, wherein the behavior of the particular advocate-agent includes one or more of a gesture of the particular advocate-agent or a speaking style of the particular advocate-agent.

18. The computer usable program product of claim 11, wherein the computer usable code is stored in a computer readable storage device in a data processing system, and wherein the computer usable code is transferred over a network from a remote data processing system.

19. The computer usable program product of claim 11, wherein the computer usable code is stored in a computer readable storage device in a server data processing system, and wherein the computer usable code is downloaded over a network to a remote data processing system for use in a computer readable storage device associated with the remote data processing system.

20. A computer system comprising one or more processors, one or more computer-readable memories, and one or more computer-readable storage devices, and program instructions stored on at least one of the one or more storage devices for execution by at least one of the one or more processors via at least one of the one or more memories, the stored program instructions comprising:
program instructions to receive one or more alternatives, each of the one or more alternative having an associated weight;
program instructions to generate an advocate-agent corresponding to each of the one or more alternatives, each advocate-agent being configured to advocate for the corresponding alternative;
program instructions to determine an interaction characteristic for each advocate-agent based on the associated weight and an engagement model;
program instructions to receive an engagement metric associated with each advocate-agent, the engagement metric being indicative of an attention level of a user towards the particular alternative associated with the particular advocate-agent; and
program instructions to adjust the interaction characteristic of a particular advocate-agent based upon the engagement metric associated with the particular advocate-agent according to the engagement model.
